(19) United States
(12) Patent Application Publication
(10) Pub. No.: US 2002/0127635 A1
(43) Pub. Date: Sep. 12, 2002

(54) Method of complexing a protein by the use of a dispersed system and proteins thereof

(76) Inventors: Sathyamangalam V. Balasubramanian, Amherst, NY (US); Robert M. Straubinger, Amherst, NY (US)

Correspondence Address: Michael L. Goldman, NIXON PEABODY LLP, Clinton Square, P.O. Box 31051, Rochester, NY, 14603, US

(21) Appl. No.: 10/000,226

(22) Filed: Nov. 30, 2001

Related U.S. Application Data
(60) Provisional application No. 60/250,283, filed on Nov. 30, 2000.

(51) Int. Cl. C12P 21/06; C12N 9/36

(52) U.S. Cl. 435/68.1; 435/206

(57) ABSTRACT

A method for complexing a protein in a dispersed medium, includes: a) providing a protein, b) altering the conformational state of the protein to expose hydrophobic domains therein, c) binding a stabilizer to the exposed hydrophobic domains, and d) at least partially reversing the alteration to associate at least a portion of the protein with the stabilizer. A pharmaceutically effective stabilized protein dosage wherein from less than about 1% to greater than about 90% of the protein is associated by a stabilizer is also provided.

### BACKGROUND OF THE INVENTION

This application claims the benefit of U.S. Provisional Patent Application Serial No. 60/250,283, filed Nov. 30, 2000 (which is hereby incorporated by reference in its entirety).

1. Field of the Invention

The present invention relates to a method for engineering the complexation of a protein by using a dispersed system. In particular, the present invention relates to engineering the complexation/association by adding a stabilizer or excipient to a protein under conformationally altering conditions, including unfolding, to enhance the hydrophobic interaction and translocation of the protein in the dispersed system.

2. Description of the Related Art

Advances in protein engineering have led to the large scale production of proteins and peptides for pharmaceutical purposes. However, for many proteins, the preservation of higher order structure, such as secondary, tertiary and quaternary conformation, is necessary to retain activity. The formulation of such suitable protein and peptide based pharmaceuticals is largely an unsolved problem. Proteins undergo physical and chemical instability, and these instabilities present unique difficulties in the production, formulation, and storage of protein pharmaceuticals (Ahern, T. J., and Manning, M. C. (1992) in Pharmaceutical Biotechnology (Borchardt, R. T., Ed.) pp 550, Plenum Press, New York; Balasubramanian., S. V., Breunn, J. A., and Straubinger, R. M. (2000) Pharmaceutical Research 17, 343-349, which are hereby incorporated by reference in their entirety). Denaturation, aggregation, and precipitation are frequent manifestations of physical instability.

Other pharmaceutical concerns of the protein products are shorter half-life and immune response following prolonged use of the drug (Ahern, T. J., and Manning, M. C. (1992) in Pharmaceutical Biotechnology (Borchardt, R. T., Ed.) pp 550, Plenum Press, New York, which is hereby incorporated by reference in its entirety). Delivery vehicles such as liposomes have been explored to improve stability, to prolong the circulation time and to alter the immunogenecity issues (Balasubramanian., S. V., Breunn, J. A., and Straubinger, R. M. (2000) Pharmaceutical Research 17, 343-349, which is hereby incorporated by reference in its entirety). It is known that when liposomes are added to proteins, the stability of proteins are improved since liposomes help reduce the amount of aggregation of the protein. However, the liposomes typically complex with only a small percentage of the total protein. Accordingly, the pharmaceutical developments of such delivery vehicles are hampered by poor association with proteins.

Thus, there is a need for suitable protein and peptide based pharmaceuticals having improved stability during processing and storage conditions; increased dosage spacing by increasing bioavailability, thus reducing cost and patient discomfort; easy handling; and improved delivery to the site of vascular damage. The present invention is directed to overcoming these and other deficiencies in the art by providing a methodology to engineer a complex between a protein and a dispersed system based delivery vehicle.

### SUMMARY OF THE INVENTION

The present invention provides a method for complexing a protein in a dispersed medium, including: a) providing a protein, b) altering the conformational state of the protein to expose hydrophobic domains therein, c) binding a stabilizer to the exposed hydrophobic domains, and d) at least partially reversing the alteration to associate at least a portion of the protein with the stabilizer.

The present invention also provides a pharmaceutically effective stabilized protein dosage wherein from less than about 1% to greater than about 90% of the protein is associated, including encapsulation, with the stabilizer.

These and other aspects of the present invention will become apparent upon a review of the following detailed description and the claims appended thereto.

### BRIEF DESCRIPTION OF THE DRAWINGS

FIG. 1(a) shows the temperature dependent changes in the secondary structure of lysozyme in ethanol-water mixtures by plotting ellipticity at 220 nm and 268 nm as a function of temperature. FIG. 1(b) shows the temperature dependent changes in the tertiary structure of lysozyme in ethanol-water mixtures by plotting ellipticity at 220 nm and 268 mu as a function of temperature.

FIG. 2 is a plot of the % change in the ANS complex formation as a function of temperature.

FIG. 3 is a ribbon diagram of the three dimensional structure of lysozyme.

### DETAILED DESCRIPTION OF PREFERRED EMBODIMENTS

The present invention relates to a method for engineering the complexation of protein with a dispersed system and the complexed proteins prepared therefrom. A dispersed system is considered any system having a hydrophobic interior and a hydrophillic exterior. A stabilizer or excipient is added at the desired stage during an alteration in the conformational state of the protein, for example, it is added to a partially folded protein under controlled unfolding conditions. In accordance with the present invention, the conformational state of a protein is altered to expose the hydrophobic domains. The hydrophobic domains of the protein are exposed to enhance the ability of the stabilizer to associate with the protein. Association includes encapsulation. Unfolding is a preferred mechanism for altering the conformational state. The complexation is engineered to enhance the hydrophobic interaction and translocation of the protein in the dispersed system in an effort to increase the association efficiency of the protein. Activity is retained by the preservation of higher order structure of the protein, such as secondary, tertiary and quaternary conformation. In this manner, problems of physical and chemical instability such as denaturation, aggregation, and precipitation can be overcome leading to improvements in the production, formulation, and storage of protein pharmaceuticals.

Experimentally, the secondary and tertiary structures of the protein are monitored under conformational altering conditions, which include unfolding. The exposure of hydrophobic domains is confirmed by the binding of a specific fluorescence probe to the exposed hydrophobic domains. In this manner, such experiments are used to identify specific conformational states of the protein with exposed hydrophobic domains. Upon exposure of the desired hydrophobic domain, the stabilizer is added.

The present invention enables the liposome association of from less than about 1% to greater than about 90% of the lysozyme protein, preferably above about 1%, more preferably above about 3%, more preferably from about 3% to about 90%, and most preferably from about 80% to about 90% of the protein. It is understood that the % association, including encapsulation, is related in part to the size of the protein and the specific % association for a given protein will vary accordingly with different size proteins.

In accordance with the present invention, a methodology is presented to engineer a complex between the protein and the dispersed system based delivery vehicle which overcomes problems typically associated with shorter half-life and immune response following prolonged use of a drug by improving stability and prolonging the circulation time of the drug. In particular, the present invention solves the pharmaceutically related problems stemming from the use of liposomes as delivery vehicles generally attributed to poor association of proteins.

Since the process of denaturation is related to conformational changes of the protein, such as unfolding at the molecular level, our approach was to analyze protein unfolding in detail, and apply novel methods at key steps in the process. Based on experiments, we developed a methodology for the complexation of protein pharmaceuticals with dispersed systems. According to the methodology, we subject the protein to conditions which change the conformation of the protein to expose its hydrophobic domains and then associate at least a portion of the protein with a stabilizer. The present formulation strategy exploits the properties of the intermediate structures. The first step is to form “structured” intermediate states using alteration in the conformational state such as controlled unfolding of the protein. Conditions are controlled carefully, enabling the exposure of domains that permit interaction with the excipient. The second step is to add the stabilizing excipient (in this case, pre-formed unilamellar liposomes), to bind to the intermediates.

Changing the conformational state, such as unfolding, of the protein to expose its hydrophobic domains is possible by both chemical and/or physical perturbants. Physical perturbants include but are not limited to thermal and pressure changes. Chemical perturabants include but are not limited to organic solvents, urea, buffers with acidic pH, guandinium hydrocholoride. Several organic solvents are compatible with protein, including alcohols such as methanol, ethanol, glycerol, ethylene glycol, and the like. The present invention is applicable to any method suitable for changing the conformational state of the protein to expose its hydrophobic domains, unfolding being preferred. The use of solvents as a perturbant for the complex formation has been chosen as an example, however, the present invention is not limited to this method alone. For example, the use of solvents in combination with heat will typically expedite the changing of the conformational state of the protein.

Lysozyme was used as an example of a protein applicable to the methods of the present invention to investigate the use of a solvent for the complex formation in a dispersed system. The present invention is not limited by the choice of protein, any protein would be applicable, including for example, biopolymers composed of natural and unnatural amino acids, and multi-domain proteins. Lysozyme was chosen as a representative protein for the following reasons. Lysozyme is a hydrophilic protein and its spontaneous encapsulation in neutral liposomes is limited. Further, the thermal stress of the protein in aqueous system do not generate intermediate structures but such structures are observed in ethanol-water mixtures. Lysozyme is a bacteriolytic protein is under investigation as a therapeutic agent for AIDS (Tavio, M., Nasti, G., Simonelli, C., Vaccher, E., De Paoli, P., Giacca, M., and Tirelli, U. (1998) Eur J Cancer 34, 1634-1637; Lunardi-Iskandar, Y., Bryant, J. L., Blattner, W. A., Hung, C. L., Flamand, L., Gill, P., Hermans, P., Birken, S., and Gallo, R. C. (1998) Nat Med 4, 428-434; Witzke, O., Hense, J., Reinhardt, W., Reiner, C., Hoermann, R., and Philipp, T. (1997) Eur J Med Res 2, 155-158, which are hereby incorporated by reference in their entirety). It has been shown that the transmission of HIV type I from mother to fetus in the first trimester is prevented by hcg beta subunit and lysozyme present in hcg b core preparations (Lunardi-Iskandar, Y., Bryant, J. L., Blattner, W. A., Hung, C. L., Flamand, L., Gill, P., Hermans, P., Birken, S., and Gallo, R. C. (1998) Nat Med 4, 428-434; Lee-Huang, S., Huang, P. L., Sun, Y., Kung, H. F., Blithe, D. L., and Chen, H. C. (1999) Proc Natl Acad Sci USA 96, 2678-2681, which are hereby incorporated by reference in their entirety). Recently, Huang et al. have shown that lysozyme obtained from other sources such as human milk and chicken egg white also possess activity against HIV-1 (Lee-Huang, S., Huang, P. L., Sun, Y., Kung, H. F., Blithe, D. L., and Chen, H. C. (1999) Proc Natl Acad Sci USA 96, 2678-2681, which is hereby incorporated by reference in its entirety). Detailed structural information is available to investigate structure-stability relationships of lysozyme.

For the purposes of the present invention, liposomes are defined as microcapsules having a hydrophobic interior and a hydrophillic exterior synthesized from lipids. Other suitable dispersed systems include micelles, detergents, and the like.

The protein was subjected to thermal stress in ethanol-water mixtures to generate intermediate structures. In water, the melting curve obtained for tertiary and secondary structural changes overlap (Tm of 74° C.). The data indicates that there are no intermediates in water but the thermal stress of the protein in ethanol-water generated intermediate structures. Such conclusions were drawn based on the melting profiles in which the Tm measured by secondary and secondary structures do not overlap. In order to investigate the effect of ethanol on unfolding of the protein, thermal denaturation studies were carried out for lysozyme in ethanol-water mixtures. Addition of ethanol as low as 5%, decreased the Tm (s) measured by secondary structural changes by 2° C. However, the Tm (t) measured by monitoring the tertiary structure decreases as the ethanol concentration was increased. At lower ethanol concentrations, such as from 0% to 10%, the midpoint of the melting profile measured by secondary and tertiary structure overlap; Tm (s) and Tm (t) were equal but as the ethanol concentration was increased, the difference between Tm (s) and Tm (t) increased. For example, in the presence of 20% ethanol, Tm (s) was found to be 72.5° C. whereas Tm (t) is 68° C. In the temperature range of from 68° C. to 72.5° C., the protein displays the properties of an intermediate state such as molten globule. This intermediate structure exposes the hydrophobic domains suitable for complex formation. The observed generation of intermediate structure may be due to the interaction of the solvent with protein. Timasheff and Inoue suggested that addition of third component to a binary (protein-water system) have important effects on the forces that stabilize the native and altered structure of the proteins (Timasheff, S. N. and Inoue, H. (1968) Biochemistry, 7:2501-2513, which is hereby incorporated by reference in its entirety). As a protein unfolds, the non-polar residues come into contact with the solvent system. In this process, the organic component used as an additive tends to cluster about these residues. Thus, in the presence of ethanol exposure of few hydrophobic residues may be thermodynamically favored.

The observed off pathway unfolding profile induced by ethanol may be due to its interaction with protein. The interaction of alcohol with proteins has been extensively investigated. Timasheff and Inoue suggested that addition of third component to a binary (protein-water) system have important effects on the forces that stabilize the native and altered structure of proteins (Timasheff, S. N., and Inoue, H. (1968) Biochemistry 7, 2501-2513, which is hereby incorporated by reference in its entirety). As a protein unfolds, the non-polar residues come into contact with the solvent system. In this process, the organic component used as an additive tends to cluster about these residues. Thus, in the presence of ethanol, exposure of few hydrophobic residues may be thermodynamically allowed and these contacts may not be favored in hydrophilic environments.

The composition of the liposomes can be modified to enhance the association with the native state preventing denaturation. Liposomes may also interact with intermediate states without altering the refolding appreciably, exerting a beneficial effect through stabilization of the intermediate states or inhibiting progression to conformations that lead to other physical instabilities, such as aggregation. Alternatively, the liposomes may act as chaperones, assisting the protein to refold to a state that resembles more closely the native structure. Finally, liposomes may guide the protein refolding to unique intermediate structures that are stabilized and active, yet different from the folding intermediates that would exist in the absence of the liposomes.

Further, the solvent based excipients may provide easier pharmaceutical processing and handling conditions during isolation, shipping, storage and administration of the therapeutic proteins. Apart from ethanol, other solvents such as glycerol, have been shown to be compatible for the stability of lysozyme (Rariy, R. V., and Klibanov, A. M. (1999) Biotechnol Bioeng 62, 704-710; Rariy, R. V., and Klibanov, A. M. (1997) Proc Natl Acad Sci USA 94, 13520-13523; Knubovets, T., Osterhout, J. J., Connolly, P. J., and Klibanov, A. M. (1999) Proc Natl Acad Sci USA 96, 1262-1267, which are hereby incorporated by reference in their entirety). In addition, other pharmaceutically acceptable solvents such as propylene glycol may be suitable candidates for the development of protein pharmaceuticals as they are well tolerated for subcutaneous administration as most of the proteins are subcutaneously administered.

### EXAMPLES

The invention will be illustrated in greater detail by the following specific examples. It is understood that these examples are given by way of illustration and are not meant to limit the disclosure or the claims to follow.

The following include experimental procedures used in the examples of the present invention.

Materials:

Hen egg-white lysozyme was purchased from Sigma (St Louis Mo.) as a crystallized dialyzed and lyophilized powder (Batch No: 57M7045). Spectroscopy grade solvents were purchased from Pharmaco Inc (Brookfield, Conn.) and used without further purification. ANS (1-anilino-8-naphthalene sulfonate), a probe of hydrophobic domains (Purohit, S., Shao, K., Balasubramanian, S. V., and Bahl, O. P. (1998) Biochemistry 36, 12355-123633; Balasubramanian, V., Nguyen, L., Balasubramanian, S. V., and Ramanathan, M. (1998) Molec. Pharmacol. 53, 926-932; Aloj, S. M., Ingham, K. C., and Edekhoch, H. (1973) Arch. Biochem.Biophys 155, 478-479, which are hereby incorporated by reference in their entirety), was purchased from Molecular Probes Inc. (Eugene Oreg.). The ethanol-water mixtures of the following examples were prepared by mixing appropriate volumes of ethanol and water as described in US Pharmacopia.

### Example 1

### Liposomal preparation and protein encapsulation

10 μmol/ml of DMPC (dimyristoyl phosphatidyl choline) dissolved in chloroform and the solvent was evaporated using a rotary evaporator to form a thin film in a round bottomed flask. MLVs (Multi lamellar vesicles) encapsulating the protein was formed by dispersing the lipid film in 20% ethanol-water mixture containing 2 mg/ml of lysozyme with gentle swirling at 70° C. The solvent was removed using nitrogen and replaced by distilled water. This procedure was used to encapsulate the intermediate structure but for the encapsulation of native states the lipid film and the protein was dispersed in water at 30° C.

Protein encapsulation was performed in accordance with the above procedure using the following solutions:

Solution A—200 μl ethanol in 800 μl water=approximately 20% ethanol

Solution B—300 μl ethanol in 700 μl water=approximately 30% ethanol

Solution C—500 μl ethanol in 500 μl water=approximately 50% ethanol

Solution D—600 μl ethanol in 400 μl water=approximately 60% ethanol

Solution E—700 μl ethanol in 300 μl water=approximately 70% ethanol

### Example 2

### Circular Dichroism experiments

CD spectra were acquired on a JASCO J715 spectropolarimeter calibrated with d10 camphor sulfonic acid. Temperature scans were acquired using a Peltier 300 RTS unit and the melting profiles were generated using software provided by the manufacturer. The spectra were acquired at heating rates of 60° C./hr and 120° C./hr: the data presented here are for 60° C./hr. For all the samples, a 10 nm cuvette was used to acquire the data. Samples were scanned in the range of from 260 nm to 200 nm for secondary structural analysis, and the protein concentration used was 20 g/ml. For near UV CD studies, the spectra were acquired in the range of from 360 nm to 270 nm, and the protein concentration used was 0.66 mg/ml. CD spectra of the protein were corrected by subtracting the spectrum of the solvent alone, and multiple scans were acquired and averaged to improve signal quality.

The refolding experiments were performed by dilution of the 70% or 30% (v/v) ethanol-water sample 10-fold with water to give 7% or 3% solvent respectively. The spectra were normalized for the effect of dilution by increasing the path length accordingly. For example, for 70% ethanol-water solution, the path length used was 1 mm and for 7% solution the path length of the cuvette was increased to 10 mm to account for the dilution. In addition, the contribution of the dilution effects were analyzed as follows; (1) the mean residue ellipticity was computed to normalize for the concentration of the protein and the path length of the quartz cuvette used; (2) the shape of the spectra also was analyzed as the shape does not vary with dilution.

### Example 3

### Fluorescence studies

Fluorescence spectra were acquired on an SLM 8000C spectrofluorometer (Urbana, Ill.). Emission spectra were acquired over the range of from 400 nm to 550 nm, using a slit width of 4 nm on the excitation and emission paths. The excitation monochromator was set at 380 nm and the emission was monitored at 482 nm. Correction for the inner filter effect was performed by appropriate procedures (Lakowicz, J. R. (1986) Principles of Fluorescence Spectroscopy, Plenum Press, New York, which is hereby incorporated by reference in its entirety). Samples were maintained at the desired temperature using a water bath (Neslab RTE 110, NESLAB Instruments Inc, Newington, N.H.). Spectra were corrected through the use of an internal reference and further processed using software provided by the manufacturer.

### Example 4

### Equilibrium folding analysis

A two-state unfolding model was applied to analyze the equilibrium unfolding data. Each unfolding curve was normalized to the apparent fraction of the unfolded form (Fₐₚₚ), using the relationship:

Fₐₚₚ=(Y_{obs}−Yₙₐₜ)/(Y_{unf}−Yₙₐₜ)

where Y_{obs} is the ellipticity (at 220 nm or 290 nm) at a given temperature, and Y_{unf} and Yₙₐₜ are the spectral values for unfolded and native structures, respectively. Y_{unf} and Yₙₐₜ are obtained by performing a linear regression analysis of the spectrum plateau region at high and low temperatures, respectively.

### Example 5—ANS binding studies:

ANS (1-anilino-8-naphthalene sulfonate) was dissolved at 1 mg/ml containing 2% ethanol, and a small volume was added to a solution of 10 M of lysozyme in water, to give a final probe concentration of 0.3M. The initial fluorescence intensity of the probe was normalized to account for the general solvent effects of ethanol on fluorescence measurements.

### Example 6

### Biological activity assay

The activity of lysozyme was determined by measuring the catalytic activity of the protein as described earlier (Rariy, R. V., and Klibanov, A. M. (1999) Biotechnol Bioeng 62, 704-710; Rariy, R. V., and Klibanov, A. M. (1997) Proc Natl Acad Sci USA 94, 13520-13523, which are hereby incorporated by reference in their entirety). The refolded protein was diluted 20 times into an assay mixture containing a prefiltered cell suspension of 0.16 mg/ml of M. lysodeikticus and the change in absorbance at 450 nm was monitored for the bacteriolytic activity of the protein. Control experiments were performed for ethanol concentrations of from 0% to 100% and the resultant data indicated that the presence of ethanol did not contribute to the activity measurement.

### Example 7

### Separation of free protein from liposome bound protein:

The liposome bound protein was separated from free protein by dextran centrifugation gradient. 0.5 ml of the liposome bound protein was mixed with 1 ml of 20% w/v of dextran and 3 ml of 10% w/v of dextran was layered over the above solution. Then 0.5 ml of water layered on the top of the above solution. The gradient was centrifuged for 35 min at 45 KPM using Beckman SW50.1 rotor.

### Example 8

### Molecular Topology of liposomal protein

The surface of the protein exposed to bulk aqueous compartment was investigated using acrylamide quenching and trypsin digestion. The fluorescence quenching by acrylamide is carried out to determine the accessibility of the protein surface to collisional quencher and would provide information on the location of the protein in liposomes.

Thermal Denaturation Studies:

Thermal stress is very often used as a denaturant to unfold protein (L. Morozova, P. Haezebrouck and F. Van Cauwelaert. Stability of equine lysozyme. I. Thermal unfolding behaviour. Biophys. Chem., 41:185-191 (1991), which is hereby incorporated by reference in its entirety) and to investigate the formation of intermediate structure(s). As unfolding of lysozyme in water follows a two-state model without the formation of intermediate(s) (M. Ikeguchi, K. Kuwajima, M. Mitani and S. Sugai. Evidence for identity between the equilibrium unfolding intermediate and a transient folding intermediate: a comparative study of the folding reactions of alpha-lactalbumin and lysozyme. Biochemistry, 25:6965-6972 (1986), which is hereby incorporated by reference in its entirety), ethanol was used in combination with thermal stress to generate intermediate structure(s).

Secondary Structure and Unfolding:

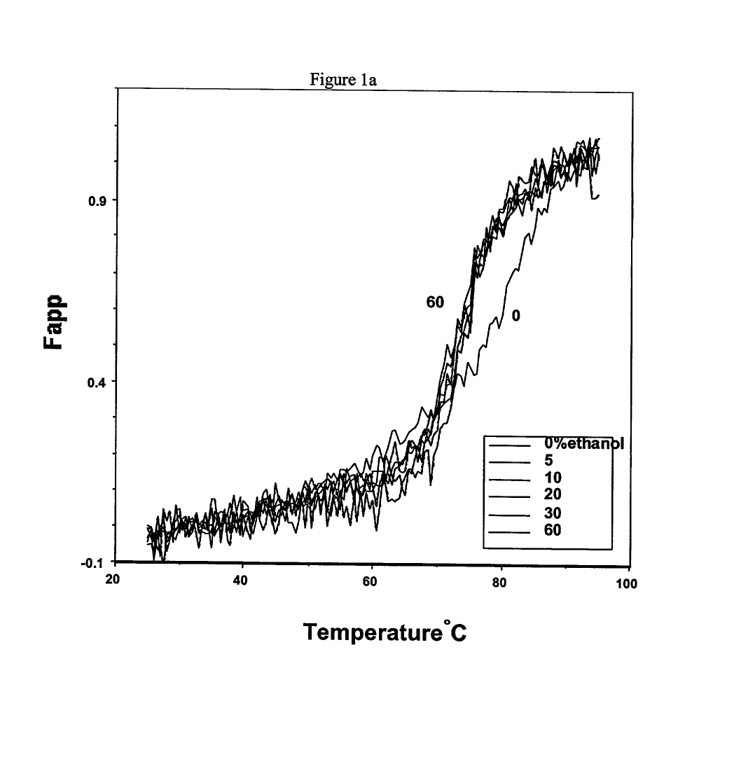

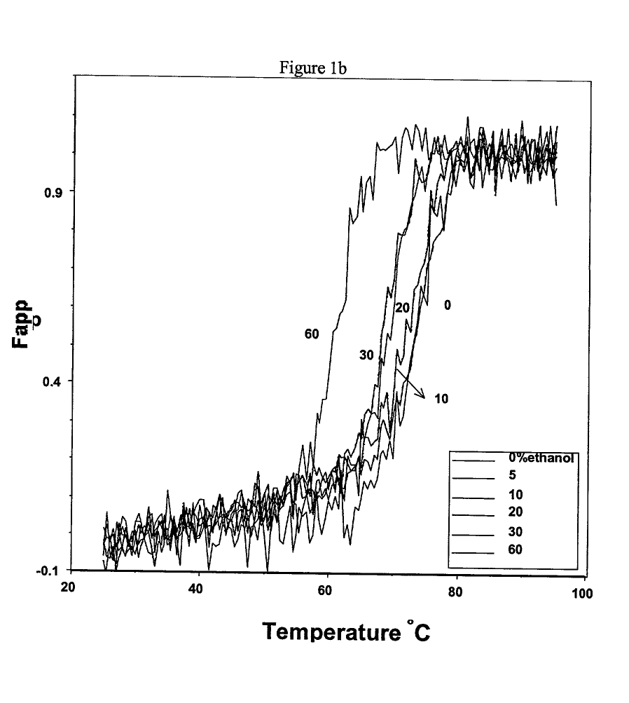

Far-UV CD spectra were acquired for lysozyme at different temperatures in various ethanol-water mixtures and a melting curve was generated using ellipticity values at 220 nm (FIG. 1a). In water, lysozyme undergoes thermal unfolding with a Tm of 74° C. The addition of ethanol (5% to 60% v/v) resulted in the reduction of the Tm to 72.5° C. The superposition test was applied for the melting curves obtained for lysozyme in the presence and in the absence of ethanol, to determine the effect of ethanol on unfolding cooperativity (Y. Luo and R. L. Baldwin. The 28-111 disulfide bond constrains the alpha-lactalbumin molten globule and weakens its cooperativity of folding. Proc. Natl. Acad. Sci. USA, 96:11283-11287 (1999), which is hereby incorporated by reference in its entirety). In water, the unfolding transition curve was broader compared to that observed in ethanol-water mixtures, suggesting a weaker cooperative transition for lysozyme in water.

Tertiary Structure and Unfolding:

The melting of lysozyme in various ethanol-water mixtures was studied by near-UV CD spectra and a melting curve was generated by plotting ellipticity values at 290 nm as a function of temperature (FIG. 1b). The Tm decreased as the ethanol concentration was increased. In water, the melting curve obtained for tertiary structural change overlaps with that observed for secondary structure, with a Tm around 74° C. This observation is consistent with previously reported results (T. Knubovets, J. J. Osterhout, P. J. Connolly and A. M. Klibanov. Structure, thermostability, and conformational flexibility of hen egg-white lysozyme dissolved in glycerol, Proc. Natl. Acad. Sci. USA, 96:1262-1267 (1999), which is hereby incorporated by reference in its entirety) suggesting that intermediate(s) are not formed during unfolding of lysozyme in water. Further, unlike secondary structural changes, the unfolding of tertiary structure in water was more cooperative, similar to that observed in ethanol-water mixtures. However, it is interesting to note that the folding characteristics of secondary and tertiary structures measured for lysozyme in ethanol-water mixtures did not overlap (FIGS. 1a and 1b). For instance, at lower ethanol concentrations (20% v/v), the midpoint of transition for the near UV CD spectrum occurred around 68.75° C. while in contrast, the transition detected by far UV CD was higher, approximately 72.5° C. In the temperature range between 68.75° C. and 72.5° C., the protein existed in a conformation where it lost its tertiary structure but has intact secondary structure. This molecular property is a characteristic of intermediate state. The cooling curve acquired for the secondary and tertiary structural changes were reversible (data not shown). Similarly, the near UV CD spectra of the unfolding of the lysozyme at higher ethanol concentrations (60% v/v) showed that the protein melted around 60° C., whereas the Tm determined by far UV CD spectra was 72.5° C. Thus, the midpoint of the melting curve for secondary and tertiary structure did not overlap, indicating the existence of intermediate structure(s).

Effects of Thermal Denaturation on the Exposure of Hydrophobic Domains:

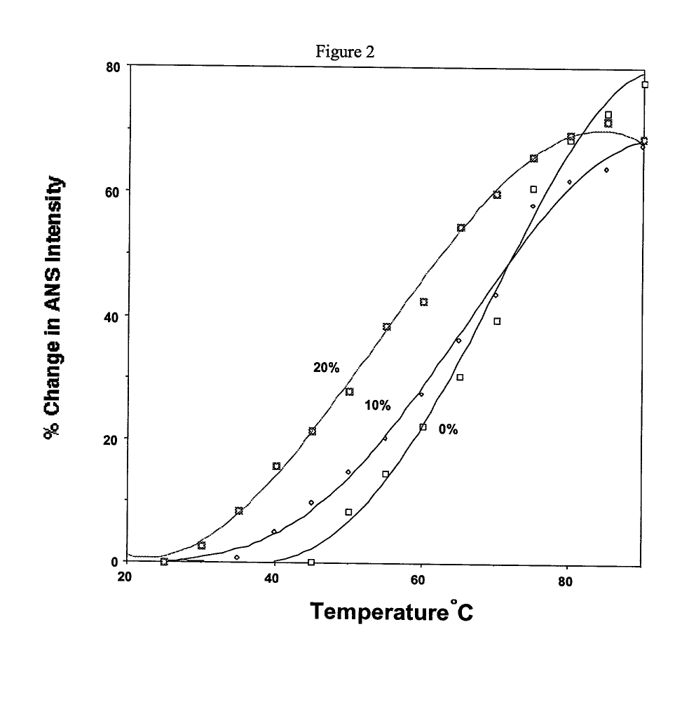

Unfolding of the protein often results in the exposure of hydrophobic domains and the binding of fluorescence probes such as 1,8 anilinonaphthalene sulfonate (ANS) have been used effectively to investigate the surface properties of the unfolding proteins (S. M. Aloj, K. C. Ingham and H. Edekhoch, Interaction of 1,8-ANS with human luteinizing hormones: A probe for subunit interactions of hcg and hlh. Arch. Biochem. Biophys., 155:478-479 (1973); V. Balasubramanian, L. T. Nguyen, S. V. Balasubramanian and M. Ramanathan. Interferon-gamma-inhibitory oligodeoxynucleotides alter the conformation of interferon-gamma, Mol. Pharmacol., 53:926-932 (1998); S. Purohit, K. Shao, S. V. Balasubramanian and O. P. Bahl. Mutants of human chorionic gonadotropin lacking N-glycosyl chains in the a subunit—mechanism for the differential action of the N-linked carbohydrates, Biochemistry, 36:12355-12363 (1998), which are hereby incorporated by reference in their entirety). The fluorescence intensity of the lysozyme-ANS complex was monitored in ethanol water mixtures as a function of temperature (FIG. 2). In water, the fluorescence intensity was unchanged in the temperature range of 25° C. to 50° C. while an increase in intensity was observed in the same temperature range for the lysozyme-ANS complex in 10% and 20% v/v ethanol-water mixtures. The data suggests that the exposure of hydrophobic domains occurs at lower temperatures in ethanol-water mixtures compared to that observed in water, possibly due to clustering of solvent molecules around the hydrophobic amino acids. In order to account for the contribution of solvent enhanced fluorescence and weak binding of the probe to the native state, the initial fluorescence intensity of the probe was normalized and the temperature dependent effects were calculated as % change rather than absolute fluorescence intensity.

Interaction of Intermediates with Liposomes:

When the protein is subjected to thermal stress in ethanol-water mixtures, the unfolding of the protein generates intermediate structures with exposed hydrophobic domains. This molecular characteristic is suitable for the liposomal encapsulation. In order to test this hypothesis we carried out the encapsulation of the protein in 20% ethanol-water mixtures at 70° C. at which the protein exist as intermediates. The solvent was removed by a nitrogen stream or by dialysis. It is appropriate to mention here that solvent removal resulted in the refolding of the protein as inferred from our equilibrium refolding experiments (K. Ramani, R. M. Straubinger and S. V. Balasubramanian, Pharm. Res., (2001) under review), which is hereby incorporated by reference in its entirety). Several control experiments including the encapsulation of the native state, i.e., protein in water at 30° C., was also carried out (Table 1). The free protein is separated from liposome bound protein by dextran centrifugation gradient and the % encapsulation of the protein was estimated by activity and fluorescence assays. It is clear from the data that the intermediate structure mediated encapsulation into the liposomes yielded higher encapsulation efficiency compared to the native state of the protein.

TABLE 1

| | % |
|---|---|
| Sample | Protein Associated |
| Native state (in water, pH 7.4 at 30° C.) | 26-30% |
| Intermediate state (in 20% ethanol-water, pH 7.4 at | 58-60% |
| 70° C.) | |

FIG. 1. Temperature dependence of secondary and tertiary structure of lysozyme in various ethanol-water mixtures.

The temperature dependent changes in secondary (FIG. 1a) and tertiary (FIG. 1b) structure of lysozyme in ethanol-water mixtures, are compared by plotting ellipticity at 220 nm and 268 nm as a function of temperature. The melting profiles were collected over the temperature range of from 25° C. to 95° C. with a heating rate of 60° C./hr at every 0.5° C. intervals. Each data point is an average of three experiments. Fₐₚₚ, the fraction of protein in the unfolded state, is calculated as described above in the experimental procedures. For secondary structure, the path length of the cuvette used was 10 mm, and the concentration of protein was 20 μg/ml. For tertiary structural measurements, the path length of the cuvette used was 10 mm, and the concentration of protein was 0.66 mg/ml.

FIG. 2. Exposure of hydrophobic domains of lysozyme in ethanol-water mixtures probed by ANS complex formation.

ANS was dissolved at high concentration in water and a small volume was added to a solution of 10 μM of lysozyme, to a final probe concentration of 0.3 μM. The samples were excited at 380 nm and the emission was monitored at 482 nm. Each data point is an average of three experiments.

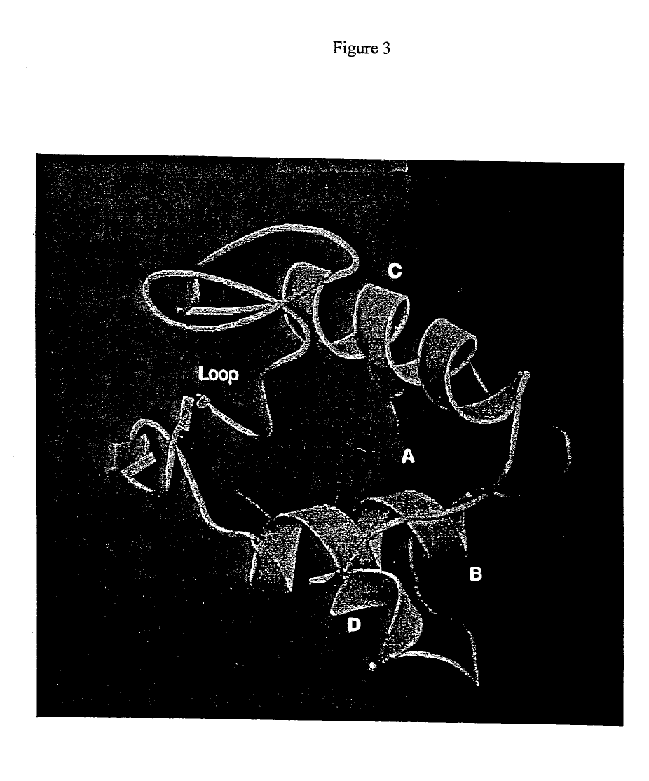

FIG. 3. Ribbon diagram of the three dimensional structure of lysozyme.

The hydrophobic core comprising of four major helices are marked as A (5-15),B (25-36, C. (88-101) and D (109-115).

While the invention has been described with preferred embodiments, it is to be understood that variations and modifications may be resorted to as will be apparent to those skilled in the art. Such variations and modifications are to be considered within the purview and the scope of the claims appended hereto.

What is claimed is:

1. A method for complexing a protein in a dispersed medium, comprising:
a) providing a protein,
b) altering the conformational state of said protein to expose hydrophobic domains therein,
c) binding a stabilizer to said exposed hydrophobic domains, and
d) at least partially reversing said alteration to associate at least a portion of said protein with said stabilizer.

2. The method of claim 1, wherein said altering comprises contacting said protein with at least one of a chemical and physical perturabant.

3. The method of claim 2, wherein said chemical perturabant comprises organic solvent, urea, buffer having an acidic pH, or guandinium hydrochloride.

4. The method of claim 2, wherein said organic solvent comprises methanol, ethanol, glycerol, or ethylene glycol.

5. The method of claim 1, wherein said altering comprises contacting said protein with an ethanol-water mixture of from about 3% to about 80%.

6. The method of claim 1, wherein said protein comprises lysozome.

7. The method of claim 2, wherein said physical perturbant comprises thermal or pressure changes.

8. The method of claim 1, wherein said stabilizer comprises liposome.

9. The method of claim 1, wherein said reversing comprises cooling.

10. The method of claim 1, wherein said reversing comprises solvent removal.

11. The method of claim 1, wherein said reversing comprises dialysis.

12. The method of claim 1, wherein said altering comprises unfolding said protein.

13. The method of claim 1, wherein said reversing comprises refolding said protein.

14. The method of claim 1, wherein said association comprises encapsulation.

15. An associated protein produced by the method of claim 1.

16. A pharmaceutically effective stabilized protein dosage wherein from less than about 1% to greater than about 90% of the protein is associated with a stabilizer.

17. The product of claim 16, further comprising a dispersed system medium.

18. The product of claim 16, wherein said stabilizer comprises liposome.

19. The product of claim 16, wherein greater than about 1% of the protein is associated.

20. The product of claim 16, wherein from greater than about 3% of the protein is associated.

21. The product of claim 16, wherein from about 80% to about 90% of the protein is associated.

22. The product of claim 16, wherein said association comprises encapsulation.

23. The product of claim 19, wherein said association comprises encapsulation.

24. The product of claim 20, wherein said association comprises encapsulation.

25. The product of claim 21, wherein said association comprises encapsulation.
